(19) United States
(12) Patent Application Publication
(10) Pub. No.: US 2013/0253628 A1
(43) Pub. Date: Sep. 26, 2013

(54) DEVICE AND METHODS FOR RENAL NERVE MODULATION

(71) Applicant: BOSTON SCIENTIFIC SCIMED, INC., Maple Grove, MN (US)

(72) Inventors: Scott R. Smith, Chaska, MN (US); Mark L. Jenson, Greenfield, MN (US)

(73) Assignee: BOSTON SCIENTIFIC SCIMED, INC., MAPLE GROVE, MN (US)

(21) Appl. No.: 13/838,356

(22) Filed: Mar. 15, 2013

Related U.S. Application Data
(60) Provisional application No. 61/614,341, filed on Mar. 22, 2012.

(51) Int. Cl. A61N 1/36

(52) U.S. Cl. CPC A61N 1/36
USPC 607/118

(57) ABSTRACT

Medical devices as well as methods for making and using medical devices are disclosed. An example medical device may include a system for nerve modulation. The system may include an elongate shaft having a proximal end region and a distal end region. A helical inflatable balloon may be coupled to the shaft. The balloon may have a proximal end, a distal end, and an outer surface. The balloon may be disposed adjacent to the distal end region of the elongate shaft. A first nerve modulation element may be attached to the balloon.

### CROSS-REFERENCE TO RELATED APPLICATIONS

This application claims priority under 35 U.S.C. §119 to U.S. Provisional Application Ser. No. 61/614,341, filed Mar. 22, 2012, the entirety of which is incorporated herein by reference.

### TECHNICAL FIELD

The present invention relates to methods and apparatuses for nerve modulation techniques such as ablation of nerve tissue or other destructive modulation technique through the walls of blood tissue.

### BACKGROUND

Certain treatments require the temporary or permanent interruption or modification of select nerve function. One example treatment is renal nerve ablation which is sometimes used to treat conditions related to congestive heart failure. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many nerves, including renal nerves, run along the walls of or in close proximity to blood vessels and thus can be accessed via the blood vessels. In some instances, it may be desirable to ablate perivascular renal nerves using a radio frequency (RF) electrode. However, such a treatment may result in thermal injury to the vessel wall at the electrode and other undesirable side effects such as, but not limited to, blood damage, clotting and/or protein fouling of the electrode. Increased cooling in the region of the nerve ablation may reduce such undesirable side effects. It is therefore desirable to provide for alternative systems and methods for intravascular nerve modulation.

### SUMMARY

The disclosure is directed to several alternative designs, materials and methods of manufacturing medical device structures and assemblies for partially occluding a vessel and performing nerve ablation.

Accordingly, one illustrative embodiment is a system for nerve modulation that may include an elongate shaft, a helical inflatable balloon on or around the elongate shaft proximate the distal end region of the elongate shaft and one or more nerve modulation elements such as electrodes attached to the balloon. When the balloon is inflated the system may have a cross-section profile equal to, smaller than or somewhat greater than the cross-sectional profile of the vessel lumen. There may be three, four, five, six or another desired number of nerve modulation elements spaced apart from each other longitudinally and circumferentially such that the treatment areas of each of the nerve modulation elements do not overlap. The nerve modulation elements may be disposed directly on the balloon surface or may be attached to the balloon by one or more spacer struts. Each nerve modulation element may have a corresponding spacer strut attaching it to the balloon. The nerve modulation elements may be positioned such that there is a gap between each element and the wall of the vessel to be treated or may be positioned against the vessel wall.

In addition to nerve modulation, the present apparatus and methods can be applied to modulation or ablation of other tissues in the body.

Some embodiments pertain to a method of performing an intravascular procedure, comprising the steps of providing a system as described herein, inflating the helical balloon to partially occlude and/or redirect blood flow, and activating the nerve modulation elements to treat and/or ablate nerve tissue proximate the nerve modulation elements.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is a schematic view illustrating a renal nerve modulation system in situ.

FIG. 2 illustrates a distal end of an illustrative renal nerve modulation system in situ.

FIG. 3A is a cross-section of the illustrative renal nerve modulation system shown in FIG. 2.

FIG. 3B illustrates schematically the renal nerve modulation system shown in FIG. 2 by combining several cross-sectional views.

FIG. 4 illustrates illustrates a distal end of an illustrative renal nerve modulation system in situ.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

While the devices and methods described herein are discussed relative to renal nerve modulation, it is contemplated that the devices and methods may be used in other applications where nerve modulation and/or ablation are desired. In some instances, it may be desirable to ablate perivascular renal nerves with deep target tissue heating. However, as energy passes from an electrode to the desired treatment region the energy may heat the fluid (e.g. blood) and tissue as it passes. As more energy is used, higher temperatures in the desired treatment region may be achieved thus resulting in a deeper lesion. However, this but may result in some negative side effects, such as, but not limited to thermal injury to the vessel wall, blood damage, clotting and/or protein fouling of the electrode. Positioning the electrode away from the vessel wall may provide some degree of passive cooling by allowing blood to flow past the electrode. However, it may be desirable to provide an increased level of cooling over the passive cooling generated by normal blood flow. In some instances, a partial occlusion catheter may be used to partially occlude an artery or vessel during nerve ablation. The partial occlusion catheter may reduce the cross-sectional area of the vessel available for blood flow which may increase the velocity of blood flow in a region proximate the desired treatment area while minimally affecting the volume of blood passing, if at all. The increased velocity of blood flow may increase the convective cooling of the blood and tissues surrounding the treatment area and reducing artery wall thermal injury, blood damage, and/or clotting. The increased velocity of blood flow may also reduce protein fouling of the electrode. The renal nerve modulation systems described herein may include other mechanisms to improve convective heat transfer, such as, but not limited to directing flow patterns with surfaces, flushing fluid from a guide catheter or other lumen, or infusing cool fluid.

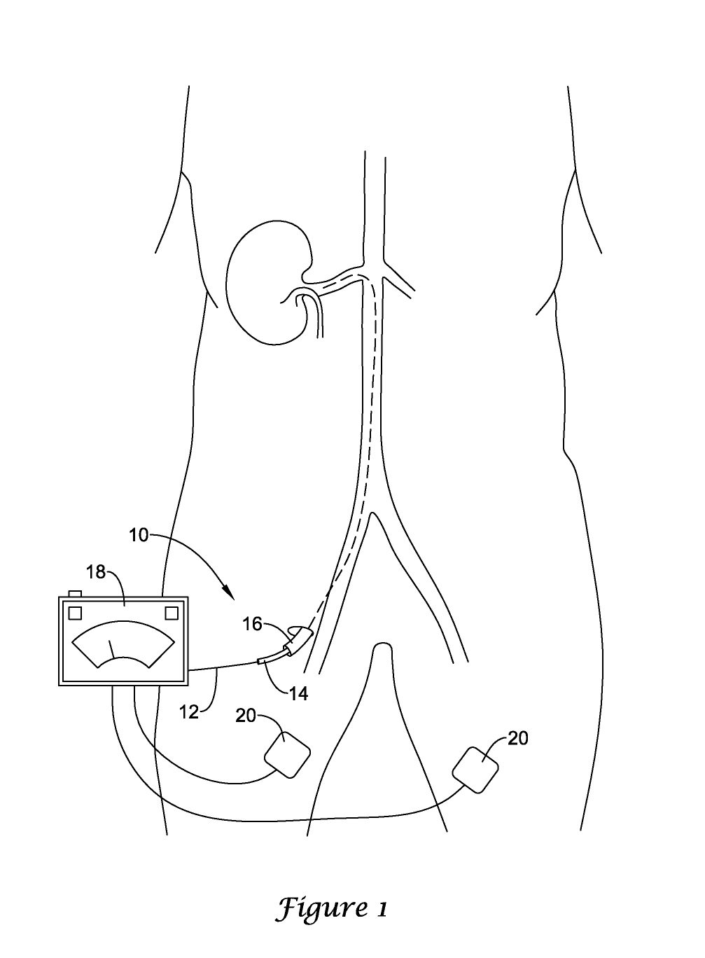

FIG. 1 is a schematic view of an illustrative renal nerve modulation system 10 in situ. System 10 may include an element 12 for providing power to an electrode disposed about and/or within a central elongate shaft 14 and, optionally, within a sheath 16, the details of which can be better seen in subsequent figures. A proximal end of element 12 may be connected to a control and power element 18, which supplies the necessary electrical energy to activate the one or more electrodes at or near a distal end of the element 12. In some instances, return electrode patches 20 may be supplied on the patient's back or at another convenient location on the patient's body to complete the circuit. The control and power element 18 may include monitoring elements to monitor parameters such as power, temperature, voltage, pulse size and/or shape and other suitable parameters as well as suitable controls for performing the desired procedure. In some instances, the power element 18 may control a radio frequency (RF) electrode. The electrode may be configured to operate at a frequency of approximately 460 kHz. It is contemplated that any desired frequency in the RF range may be used, for example, from 450-500 kHz. Lower or higher frequencies may be used, such as 10 kHz or 1000 kHz, in some cases, although the desired heating depth, catheter size, or electrical effects can limit the choice of frequency. However, it is contemplated that different types of energy outside the RF spectrum may be used as desired, for example, but not limited to ultrasound, microwave, and laser.

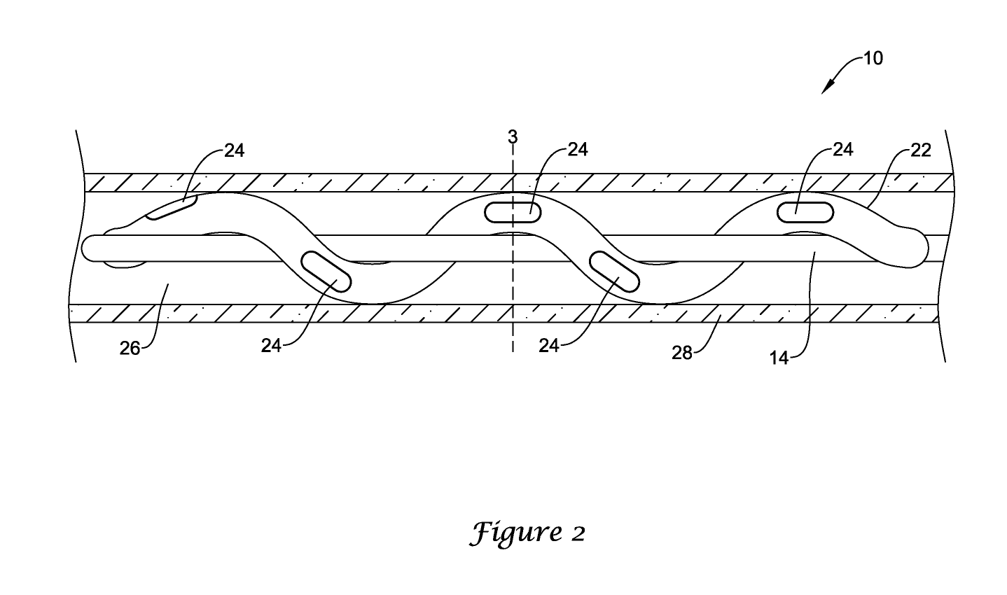

FIG. 2 is an illustrative embodiment of a distal end of a renal nerve modulation system 10 disposed within a body lumen 26 having a vessel wall 28. The system 10 may include an elongate shaft 14 having a distal end region. The elongate shaft 14 may extend proximally from the distal end region to a proximal end configured to remain outside of a patient's body. The proximal end of the elongate shaft 14 may include a hub attached thereto for connecting other treatment devices and/or providing a port for facilitating other treatments. The elongate shaft 14 may further include one or more lumens extending therethrough. For example, the elongate shaft 14 may include a guidewire lumen and/or one or more inflation lumens. The lumens may be configured in any way known in the art. For example, the guidewire lumen may extend the entire length of the elongate shaft 14 such as in an over-the-wire catheter or may extend only along a distal portion of the elongate shaft 14 such as in a single operator exchange (SOE) catheter. These examples are not intended to be limiting, but rather examples of some possible configurations. While not explicitly shown, the modulation system 10 may further include temperature sensors/wire, an infusion lumen, radiopaque marker bands, fixed guidewire tip, a guidewire lumen, external sheath and/or other components to facilitate the use and advancement of the system 10 within the vasculature may be incorporated.

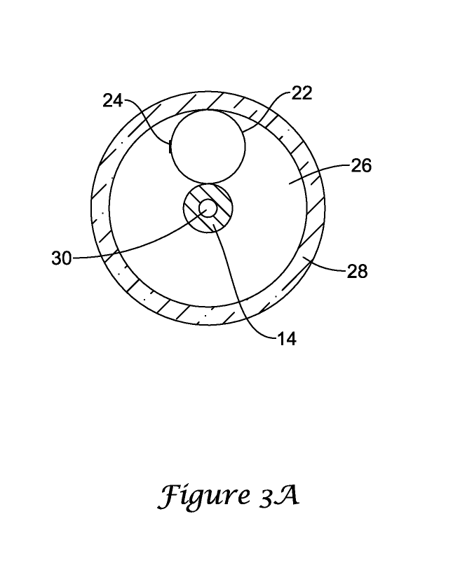

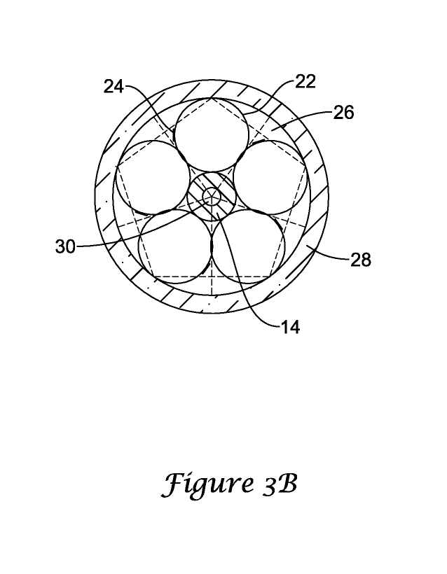

The modulation system 10 may include a balloon 22 disposed around the distal end of shaft 14. The balloon 22 may have a spiral or helical shape configured to wrap around the perimeter of the shaft 14. Disposed on balloon 22 may be one or more electrodes 24. In the embodiment shown, the one or more electrodes 24 are spaced at intervals from each other along the longitudinal axis of the shaft 14 and, as can be better seen with respect to FIGS. 3A and 3B, discussed in greater detail below, each electrode 24 is positioned with respect to the shaft 14 at a different circumferential location from its neighbors as well. In other embodiments, the number and positioning of the electrodes may be varied as desired. For example, there may be one, two, three, four, five, six, seven, eight or more electrodes and either or both of the longitudinal and circumferential positioning of the electrodes may be at a regular and repeating interval from its neighbors. In some embodiments, the positioning of the electrodes is varied as desired with irregular intervals between adjacent electrodes. In preferred embodiments, the electrodes are spaced so that the areas treated by modulation or ablation using the electrodes on the vessel wall 28 fully encircle the vessel lumen 26 while keeping the treatment areas spaced apart axially.

The spiral shape of the balloon 22 may provide controlled spacing for fluid flow between an electrode 24 and the vessel wall 28. FIG. 3A, for example, illustrates that the electrode 24 is positioned on the balloon wall at a position that, when the balloon 22 is inflated, keeps a predetermined distance between the electrode and the vessel wall. The spiral shape of the balloon 22 may also provide for a spiral path for fluid flow that may increase heat transfer away from the surface of the vessel wall in the treatment region. This may reduce negative side effects of nerve ablation, such as, but not limited to thermal injury to the vessel wall, blood damage, clotting and/or protein fouling of the electrode.

While the balloon 22 is shown as having a circular cross-section, it is contemplated the balloon 22 may have any shape or size desired. For example, the balloon may have a kidney-shaped cross-section. It is contemplated that the stiffness of the elongate shaft 14 in combination with the compliance of the balloon 22 may be modified to form modulation systems 10 for use in various vessel diameters. The balloons discussed herein, in this embodiment and in the preceding and following embodiments, are generally made from an insulating material or from a material that does not conduct electricity well, except as otherwise specifically described. Thus, current travelling between one electrode and another or between one electrode and a ground will avoid travelling through the balloon material.

The modulation system 10 may be advanced through the vasculature in any manner known in the art. For example, system 10 may include a guidewire lumen (not shown) to allow the system 10 to be advanced over a previously located guidewire. In some embodiments, the modulation system 10 may be advanced, or partially advanced, within a guide sheath such as the sheath 16 shown in FIG. 1. The balloon 22 may be deflated during introduction, advancement, and removal of the system 10. Once the distal end 30 of the modulation system 10 has been placed adjacent to the desired treatment area, the balloon 22 may be inflated to partially occlude the vessel lumen 52. Once inflated the balloon 22 reduces the cross-sectional area of the vessel lumen 26 and helps to maintain consistent spacing between the vessel wall 28 and the electrode(s) 24. The inflated balloon 22 may occupy 50% or more of the vessel lumen 26 (cross-section) over a short distance (approximately 1-2 cm) without significantly affecting the volumetric flow of blood passing the partial occlusion. The partial occlusion of the lumen 26 may increase the velocity of blood through the remaining portion of the lumen 26 which may result in an increased amount of convective cooling in the treatment region. It is further contemplated that the balloon 22 may be deflated at the treatment region to allow for longitudinal and circumferential adjustment of the modulation system 10. For example, in some instances, the modulation system 10 may be energized several different times while the elongate shaft 14 is longitudinally displaced in order to perform an ablation over a desired length.

Returning to FIG. 2, the system 10 includes one or more electrodes 24 disposed on the outer surface of the balloon 22. In some embodiments, the electrode(s) 24 may be formed of a separate structure and attached to the balloon 22. For example, the electrode(s) 24 may be machined or stamped from a monolithic piece of material and subsequently bonded or otherwise attached to the balloon 22. In other embodiments, the electrode(s) 24 may be formed directly on the surface of the balloon 22. For example, the electrode(s) 24 may be plated, printed, or otherwise deposited on the surface. In some instances, the electrode(s) 24 may be radiopaque marker bands. The electrode(s) 24 may be formed from any suitable material such as, but not limited to, platinum, gold, stainless steel, cobalt alloys, or other non-oxidizing materials. In some instances, titanium, tantalum, or tungsten may be used. It is contemplated that the electrode(s) 24 may take any shape desired, such as, but not limited to, square, rectangular, circular, oblong, etc. In some embodiments, the electrode(s) 24 may have rounded or insulated edges in order to reduce the affects of sharp edges on current density. The size of the electrode(s) 24 may be chosen to optimize the current density without increasing the profile of the modulation system 10. For example, an electrode 24 that is too small may generate high local current densities resulting in greater heat transfer to the blood and surrounding tissues. An electrode 24 that is too large may require a larger balloon 22 to carry it. It is contemplated that with a suitably flexible material, electrodes 24 of any size may be placed on the balloons 22. In some instances, the electrode(s) 24 may have an aspect ratio of 2:1 or greater (length to width). Such an elongated structure may provide the electrode(s) 24 with more surface area without increasing the profile of the modulation system 10. While the electrode(s) 24 are shown as disposed on the balloon 22, it is contemplated that in some embodiments, the electrode(s) 24 may be disposed on the surface of shaft 14. In other embodiments, a region of one, or both, of the balloon 22 and elongate shaft 14 may be made conductive. In some embodiments, the electrodes 24 may be a single electrode disposed around the entire perimeter of the balloon 22.

The balloon 22 may space the electrodes 24 a distance from the vessel wall 28 in an off-the-wall or non-contact arrangement. The balloon 22 may further maintain consistent spacing between the vessel wall 28 and the electrodes 24 such that fluid flow past the electrodes 24 may be preserved. However, in some embodiments, the balloon 22 and/or elongate shaft 14 may be arranged such that the electrodes 24 contact the vessel wall 28. While not explicitly shown, the electrodes 24 may be connected to a control unit (such as control unit 18 in FIG. 1) by electrical conductors. Once the modulation system 10 has been advanced to the treatment region, energy may be supplied to the electrodes 24. The amount of energy delivered to the electrodes 24 may be determined by the desired treatment. For example, more energy may result in a larger, deeper lesion. In some embodiments, it may be desired to achieve the hottest, deepest lesion beyond the vessel wall 28 while minimizing the temperature at the surface of the vessel wall 28. The temperature at the surface of the vessel wall 28 may be a function of the power used as well as the fluid flow through the vessel 26. In some instances, the increased velocity of fluid flow resulting from the partial vessel occlusion may allow more power to be used during treatment. While the current density traveling between, for example, electrode 24 and ground electrode 20 (shown in FIG. 1) may result in the heating of adjacent fluid and tissue, there may be negligible resistance in the electrode 24 such that the electrode 24 does not get hot.

It is contemplated that the modulation system 10 may be operated in a variety of modes. In one embodiment, the system 10 may be operated in a sequential unipolar ablation mode. The electrodes 24 may each be connected to an independent power supply such that each electrode 24 may be operated separately and current may be maintained to each electrode 24. In sequential unipolar ablation, one electrode 24 may be activated such that the current travels from the electrode 24 to the ground electrode 20. Once one area has been ablated, another electrode 24 may be activated such that current travels from the electrode 24 between the balloon 22 to the ground electrode 20 to ablate another region. In another embodiment, the system 10 may be operated in a simultaneous unipolar ablation mode. In simultaneous unipolar ablation mode, the electrodes 24 may be activated simultaneously such that current travels from each electrode 24 between the balloon 22 to the ground electrodes 20. In some instances, the electrodes 24 may each be connected to an independent electrical supply such that current is maintained to each electrode 24. In this mode, more current may be dispersed circumferentially. This may result in a more effective, deeper penetration compared to the sequential unipolar ablation mode.

In another embodiment, the system 10 may be operated in a bipolar mode. In this instance, two electrodes 24 disposed at the treatment location may be 180° out of phase such that one electrode 24 acts as the ground electrode (e.g. one cathode and one anode). As such current may flow around the elongate shaft 14 and around balloon 22 from one electrode 24 to the other electrode 24. In general, either sequential or simultaneous unipolar mode may penetrate more deeply than the bipolar mode. Because balloon 22 is generally insulating, the current density is forced around the balloons, and thus more of the current density may penetrate the vessel wall 28 and surrounding tissue. While described with respect to the illustrative embodiment of FIGS. 2-3 it is to be understood that any of the embodiments described herein may be operated in any of the above described modes.

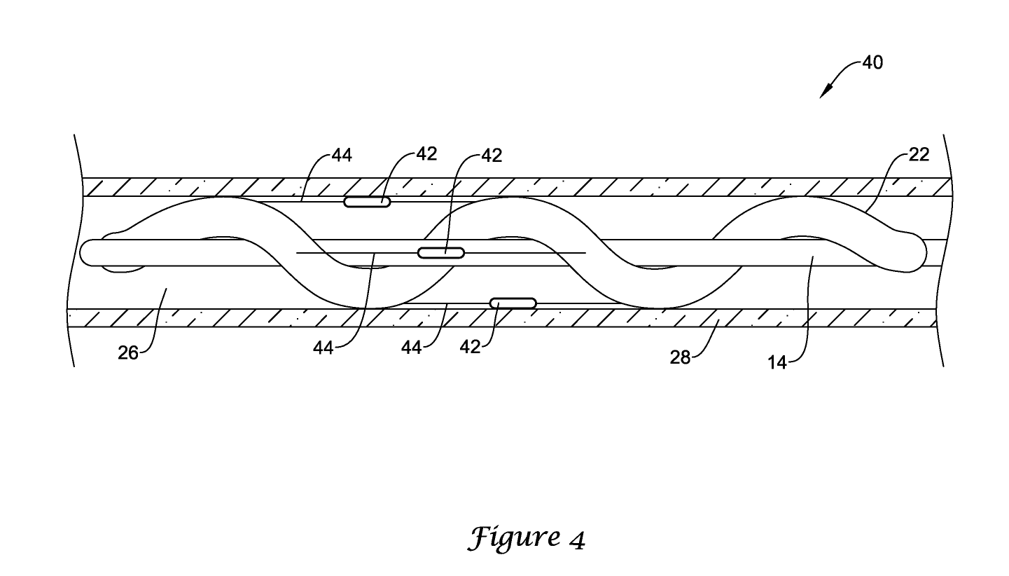

FIG. 4 is another illustrative embodiment of a distal end of a renal nerve modulation system 40 disposed within a body lumen 26 having a vessel wall 28. The system 40 may include an elongate shaft 14 having a distal end region. It is contemplated that the system 40 may incorporate the features and may use the methods described with respect to the modulation system 10 illustrated in FIGS. 2-3, above, except as otherwise described. Like the system 10 of FIG. 2, the system 40 may include an inflatable balloon 22 disposed on or adjacent to the elongate shaft 14. The balloon 22 may have a spiral shape configured to wrap around the perimeter of the elongate shaft 14 and may provide controlled spacing for fluid flow between the electrodes 42 and the vessel wall 28. The spiral shape of the balloon 22 may provide a spiral path for fluid flow thus increasing heat transfer away from the treatment region. In some embodiments, when inflated the balloon 22 may partially deform the shaft 14 to induce a corresponding spiral in the elongate shaft 14.

In the embodiment of FIG. 4, the one or more electrodes 42 are not directly on the balloon 22 or shaft 14. Instead, the electrodes 42 are on spacer struts 44. Each electrode 42 may be on a separate spacer strut 44 or more than one electrode 42 may be on a spacer strut. A first end of a spacer strut 44 may be attached to the helical balloon 22 at a first location and a second end of the spacer strut 44 may be attached to the helical balloon at a second location. In the embodiment illustrated, the first and second locations of attachment are at the same circumferential position with respect to shaft 14 and are spaced longitudinally from each other. The first and second locations are also on consecutive loops of the helical balloon 22. In the embodiment illustrated, the spacer strut 44 positions the electrode 42 against the vessel wall 28 centrally between the consecutive loops. In other contemplated embodiments, the spacer strut 44 positions the electrode 42 a predetermined distance from the vessel wall and may space the electrode closer to one loop as desired. Each spacer strut 44 may also act as a conductor to supply power to the electrodes 42. The electrodes may be positioned at regular, repeated intervals. In the side view of FIG. 4, three electrodes 42 are visible and are spaced at 90 degree intervals. A fourth electrode 42 (not shown) is obscured behind the shaft 14 and is spaced 90 degrees from the rightmost electrode shown in the figure. It can be appreciated that there are other variations of this embodiment having a fewer or greater number of electrodes. The electrodes 42 may other incorporate the features described above with respect to electrodes 42.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. A system for nerve modulation, comprising an elongate shaft having a proximal end region and a distal end region;
a helical inflatable balloon having a proximal end, a distal end, and an outer surface, the balloon being disposed adjacent to the distal end region of the elongate shaft; and
a first nerve modulation element attached to the balloon.

2. The system of claim 1, wherein the balloon is wound around the distal end region of the elongate shaft.

3. The system of claim 1, wherein the balloon is configured to deform the elongate shaft when inflated.

4. The system of claim 1, wherein the system has a cross-sectional profile smaller than a cross-section of a target vessel when the balloon is inflated.

5. The system of claim 1, wherein the system has a cross-sectional profile approximately equal to or larger than a cross-section of a target vessel when the balloon is inflated.

6. The system of claim 1, further comprising a second nerve modulation element.

7. The system of claim 1, wherein the first nerve modulation element is an electrode.

8. The system of claim 1, wherein the first nerve modulation element has an oblong shape.

9. The system of claim 1, wherein the elongate shaft further includes at least one inflation lumen.

10. The system of claim 1, wherein the first nerve modulation element is attached to a support strut having a first end and a second end, wherein the first end of the support strut is attached to the outer surface of the balloon at a first location and the second end of the support strut is attached to the balloon at a second location different from the first location.

11. The system of claim 10, wherein the support strut extends parallel to the shaft.

12. The system of claim 10, wherein the balloon comprises a first helical loop and a second helical loop, the second helical loop being adjacent to the first helical loop, and wherein the first end of the support strut is attached to the first helical loop and the second end of the support strut is attached to the second helical loop.

13. A method of nerve modulation, the method comprising:
providing a system for nerve modulation, the system comprising:
an elongate shaft having a proximal end region and a distal end region,
a helical inflatable balloon having a proximal end, a distal end, and an outer surface, the balloon being disposed adjacent to the distal end region of the elongate shaft; and
a first nerve modulation element attached to the balloon;
inserting the distal end region of the system percutaneously to an region of interest; and
activating the first nerve modulation element.

14. The method of claim 13, wherein activation of the first nerve modulation element includes activating the first nerve modulation element in a sequential unipolar mode.

15. The method of claim 13, wherein activation of the first nerve modulation element includes activating the first nerve modulation element in a simultaneous unipolar mode.

16. The method of claim 13, wherein activation of the first nerve modulation element includes activating the first nerve modulation element in a bipolar mode.

17. A medical device for nerve modulation, comprising
a catheter shaft having a distal end region;
an expandable member helically disposed along the distal end region; and
one or more electrodes coupled to the expandable member.

18. The medical device of claim 17, further comprising a power supply electrically connected to the one or more electrodes.

19. The medical device of claim 17, wherein the one or more electrodes includes a first electrode and a second electrode, and further comprising a controller configured to activate the first electrode independently from the second electrode.

20. The medical device of claim 17, wherein the expandable member includes an expandable balloon.
